(19) United States
(12) Patent Application Publication
(10) Pub. No.: US 2024/0418669 A1
(43) Pub. Date: Dec. 19, 2024

(54) ELECTROCHEMICAL BIOMOLECULE-FUNCTIONALIZED SENSOR DEVICE AND METHODS OF MAKING AND USING THE SAME

(71) Applicants: BOARD OF REGENTS OF THE NEVEADA SYSTEM OF HIGHER EDUCATION, ON BEHALF OF THE UNIV. OF NEVADA, Reno, NV (US); NEVADA RESEARCH & INNOVATION CORPORATION, Reno, NV (US)

(72) Inventors: Manoranjan Misra, Reno, NV (US); Subhash C. Verma, Reno, NV (US); Md Al Mahmnur Alam, Reno, NV (US)

(21) Appl. No.: 18/704,313

(22) PCT Filed: Oct. 27, 2022
(86) PCT No.: PCT/US2022/048079

§ 371 Date: Apr. 24, 2024

Related U.S. Application Data
(60) Provisional application No. 63/263,217, filed on Oct. 28, 2021.

(51) Int. Cl. G01N 27/327; C25D 9/02; C25D 11/26; G01N 33/543; G01N 33/569

(52) U.S. Cl. CPC G01N 27/3278; C25D 9/02; C25D 11/26; G01N 27/3276; G01N 33/5438; G01N 33/56983; G01N 2333/165; G01N 2469/10

(57) ABSTRACT

Disclosed herein are embodiments of an electrochemical biomolecule-functionalized sensor device for rapidly determining whether a subject has, or is at risk of developing, a disease. In particular embodiments, the device embodiments are used to determine if a subject has a disease, such as COVID-19. The device embodiments comprise an electrode component that comprises functionalized nanotubes that are associated with a coating comprising a surface binding agent and a biomolecule. The coating provides the ability to specifically bind biological indicators present in a biological sample with rapid detection times.

### FIELD

The present disclosure is directed to electrochemical sensor device embodiments that are modified with biomolecules to facilitate rapid disease detection and methods of making and using such sensor device embodiments.

### BACKGROUND

Sensor devices for detecting diseases have been developed in the field of diagnostics; however, currently available devices suffer from drawbacks that detract from their use in non-invasive diagnostic methods, such as poor detection limits and/or unreliable responses, and/or that produce false-positive or false-negative results due to insufficient sensitivity profiles. And, such devices also require assembly methods or complex components/configurations that result in high manufacturing costs and the inability to produce single-use devices. Particularly in the last year, COVID-19 has impacted the medical field as a novel coronavirus with a greater spread rate than influenza. The ability to rapidly detect viruses, such as the coronavirus that causes COVID-19 proves valuable in preventing widespread exposure of disease and can assist in contact tracing, which mitigates the chance of an epidemiological crises. There exists a need in the art of diagnostics and disease control for new devices that exhibit improved detection limits, more reliable responses during use, quicker detection time periods, and that can be made in a cost-effective manner, particularly for point-of-use care.

### SUMMARY

Disclosed herein are embodiments of an electrode, comprising a plurality of functionalized nanotubes on a support, wherein the functionalized nanotubes comprise metal oxide-based nanotubes that are associated with a coating comprising a surface binding agent and biomolecule.

Also disclosed herein are embodiments of a substrate-based platform comprising an electrode embodiment of the present disclosure and a substrate physically attached to the support of the electrode.

In yet additional embodiments, a sensor device is disclosed, which is an electrochemical biomolecule-functionalized sensor device, comprising: a working electrode component, comprising an electrode embodiment disclosed herein or a substrate-based platform embodiment disclosed herein; reference electrode; a counter electrode; and a potentiostat.

Method embodiments also are described, including embodiments of a method, comprising: applying a voltage to an electrochemical biomolecule-functionalized sensor device as described herein; exposing the electrochemical biomolecule-functionalized sensor device to a biological sample; and sensing a change in current produced by the electrochemical biomolecule-functionalized sensor device after being exposed to the biological sample. Additional method embodiments concern a method making an electrode as described herein, comprising: performing a first anodization of the support to obtain the metal oxide-based nanotubes formed thereon; performing a second anodization of the metal oxide-based nanotubes to increase the length of the metal oxide-based nanotubes; depositing the surface binding agent on the metal oxide-based nanotubes to form a layer of the surface binding agent on surfaces of metal oxide-based nanotubes; and depositing a solution comprising the biomolecule onto the layer of the surface binding agent.

The foregoing and other objects and features of the present disclosure will become more apparent from the following detailed description, which proceeds with reference to the accompanying figures.

### BRIEF DESCRIPTION OF THE DRAWINGS

FIG. 1 is a schematic illustration of an exemplary method embodiment used to fabricate an electrode comprising a nanotube array.

FIGS. 2A-2C show a photographic images of representative electrode embodiments described herein, including (i) an electrode comprising nanotubes functionalized with a coating comprising a biomolecule (e.g., an antibody) and a surface binding agent (e.g., aminopropyl triethoxysilane or “APTES”), wherein the nanotubes are amorphous TiO₂ (FIG. 2A); (ii) an electrode comprising nanotubes functionalized with a coating comprising a biomolecule (e.g., an antibody) and a surface binding agent (e.g., APTES), wherein the nanotubes are anatase TiO₂ (FIG. 2B); and (iii) an electrode comprising nanotubes functionalized with a coating comprising a biomolecule (e.g., an antibody) and a surface binding agent (e.g., polyaniline), wherein the nanotubes are anatase TiO₂ (FIG. 2C).

FIG. 3 is an illustration showing photographs of a plurality of nanotubes before and after electrodeposition of polyaniline (“PANi”) on surfaces of the nanotubes.

FIGS. 4A-4C are schematic illustrations of method embodiments used to make a coating of a surface binding agent and a biomolecule having a suitable sensing orientation on a plurality of nanotubes to provide representative electrode embodiments described herein; FIG. 4A shows a method for forming the electrode illustrated in FIG. 2A; FIG. 4B shows a method for forming the electrode illustrated in FIG. 2B; and FIG. 4C shows a method for forming the electrode illustrated in FIG. 2C.

FIG. 5 is a photograph of an exemplary electrochemical biomolecule-based sensor device comprising an exemplary working electrode and other exemplary components used for sensing.

FIG. 6 is a photograph of an SEM image of a nanotube array at a scale bar of 500 nm made according to a method embodiment described herein, such as the method illustrated by FIG. 1.

FIGS. 7A and 7B show results obtained using a electrochemical biomolecule-based sensor device embodiment described herein, wherein FIG. 7A is a graph of current (uA) as a function of time (seconds) showing the amperometric response obtained after contacting the device with 10, 50, and 100 nM of an SARS-COV-2 spike protein; and FIG. 7B is a plot of sensor response as a function of concentration (nM) calculated from the generated current.

FIGS. 8A and 8B show results obtained using a electrochemical biomolecule-based sensor device embodiment described herein, wherein FIG. 8A is a graph of current (uA) as a function of time (seconds) showing amperometric response obtained after contacting the device with 0.5, 2.5 and 5.3 ng/μL of an SARS-COV-2 spike protein; and FIG. 8B is a plot of sensor response as a function of concentration (ng/μL) calculated from the generated current.

FIGS. 9A-9C are graphs of current (uA) as a function of time (seconds) showing results obtained from analyzing the amperometric response of different electrochemical biomolecule-based sensor device embodiments after contacting a working electrode of the device with SARS-COV-2 spike protein and focal glomerulosclerosis protein, wherein FIG. 9A shows results for a device comprising the electrode of FIG. 2A; FIG. 9B shows the results for a device comprising the electrode of FIG. 2B, and FIG. 9C shows the results for a device comprising the electrode of FIG. 2C; all used at a bias voltage of −0.6 V.

FIGS. 10A-10C are graphs of current (μA) as a function of time (seconds) showing results obtained from analyzing the amperometric response of different electrochemical biomolecule-based sensor device embodiments after contacting a working electrode of the device with SARS-COV-2 spike protein and focal glomerulosclerosis protein; FIG. 10A shows results for a device comprising the electrode of FIG. 2A; FIG. 10B shows the results for a device comprising the electrode of FIG. 2B; and FIG. 10C shows the results for a device comprising the electrode of FIG. 2C; all used at a bias voltage of −0.5 V.

FIGS. 11A and 11B are graphs of current (μA) as a function of time (seconds) (FIG. 11A) and sensor response (FIG. 11B) showing results obtained from analyzing the amperometric response of a sensor device embodiment after contacting a working electrode of the device with different samples; FIG. 11A shows the chronoamperometic (CA) curves obtained after contacting (using a bias voltage of-0.5 V and a scan time of 150 seconds; with sample injection at 15th second) a working electrode of the device with samples of saliva (as control), SARSCOV2, HCoV-OC43, and Adenovirus; and FIG. 11B shows a summary of the sensor response for each sample.

FIGS. 12A-12E show additional results obtained from using a sensor device embodiment with samples selected from saliva (as control), SARSCOV2, HCoV-OC43, and Adenovirus, wherein FIG. 12A shows the cyclic voltammograms (scan rate of 50 mV/s) obtained from contacting the sensor device with the samples; FIG. 12B shows a Nyquist plot with fitted data based on the modified equivalent circuit used for the saliva (control) sample in PBS solution; FIG. 12C shows the Nyquist plot for the HCoV-OC43 sample in PBS solution; FIG. 12D shows the Nyquist plot for the Adenovirus sample in PBS solution; and FIG. 12E shows the Nyquist plot for the SARSCOV2 sample in PBS solution.

### DETAILED DESCRIPTION

I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and compounds similar or equivalent to those described herein can be used in the practice or testing of the present disclosure, suitable methods and compounds are described below. The compounds, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Ratios, concentrations, amounts, and other numerical data may be expressed herein in a range format. It is to be understood that such a range format is used for convenience and brevity, and thus, should be interpreted in a flexible manner to include not only the numerical values explicitly recited as endpoints of the range, but also to include all the individual numerical values or sub-ranges encompassed within that range. Furthermore, not all alternatives recited herein are equivalents.

For any formulas provided herein, the symbol “ [symbol] ” is used to indicate a bond disconnection in any abbreviated structures/formulas provided herein. A person of ordinary skill in the art recognizes that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. Any functional group disclosed herein and/or defined above can be substituted or unsubstituted, unless otherwise indicated herein.

To facilitate review of the various embodiments of the disclosure, the following explanations of specific terms are provided.

Aliphatic: A hydrocarbon group having at least one carbon atom to 50 carbon atoms (C1-50), such as one to 25 carbon atoms (C1-25), or one to ten carbon atoms (C1-10), and which includes alkanes (or alkyl), alkenes (or alkenyl), alkynes (or alkynyl), including cyclic versions thereof, and further including straight-and branched-chain arrangements, and all stereo and position isomers as well.

Alkoxy: —O-aliphatic, such as —O-alkyl, —O-alkenyl, —O-alkynyl; with exemplary embodiments including, but not limited to, methoxy, ethoxy, n-propoxy, isopropoxy, n butoxy, t butoxy, sec butoxy, n-pentoxy (wherein any of the aliphatic components of such groups can comprise no double or triple bonds, or can comprise one or more double and/or triple bonds).

Amorphous Form: A non-crystalline form of a metal oxide in which atoms and/or molecules of the metal oxide are not organized in a definite lattice pattern.

Amino-Containing Compound: A compound (including single compound species or polymeric compound species) comprising at least one amine group, wherein the amine group is not tri-substituted and thus can form a covalent bond with a functional group (e.g., a carboxylic acid group) of a biomolecule.

Amino-Silane Compound: A compound comprising an amine group bound to a silicon atom, wherein the silicon atom is further bound to three other groups, such as alkoxy groups.

Anatase Form: A metastable mineral form of a metal oxide, such as titanium dioxide.

Biological Indicator: A substance of interest that is detected, measured, and/or identified using an electrochemical biomolecule-functionalized sensor device according to the present disclosure. In particular disclosed embodiments, the biological indicator is a species associated with a particular physiological disease or condition, such as an antigen, a bacterium, a virus, or a cellular component thereof.

Blocking Agent: A compound or molecule that can form a bond with a surface binding agent and that does not bind a biological indicator.

Carboxylic Acid-Containing Compound: A compound (including single compound species or polymeric compound species) comprising at least one carboxylic acid group (—C(O)OH), wherein the carboxylic acid group is capable of forming a covalent bond with a functional group (e.g., an amine group) of a biomolecule.

Functionalized Nanotubes: A plurality of nanotubes that are associated with a coating comprising a surface binding agent and a biomolecule. In some embodiments, the plurality of nanotubes can be functionalized with the coating such that the coating is positioned along a top surface of the plurality. In some embodiments, individual nanotubes of the plurality can be functionalized with the coating such that the surface (including external and/or interior surfaces) of one or more of the individual nanotubes are associated with the coating.

Haloacetyl-Containing Group: A compound (including single compound species or polymeric compound species) comprising at least one haloacetyl group having a core structure

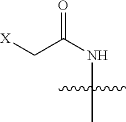

(wherein X is a halogen selected from I, Br, F, or Cl), wherein the haloacetyl group is capable of forming a covalent bond with a functional group (e.g., a thiol) of a biomolecule. In some embodiments, the haloacetyl group can be functionalized, such as at the methylene carbon atom between X and the carbonyl group.

Maleimide-Containing Group: A compound (including single compound species or polymeric compound species) comprising at least one maleimide group having a core structure

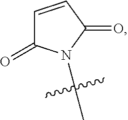 [SMILES: *N1C(=O)C=CC1=O]

wherein the maleimide group is capable of forming a covalent bond with a functional group (e.g., a thiol) of a biomolecule. In some embodiments, the maleimide group can be functionalized, such as at the sp2 carbon atoms forming the olefin present in the maleimide group.

Nanotube: A nanometer-scale tube-like structure that typically is hollow.

Pyridyldithiol-Containing Group: A compound (including single compound species or polymeric

compound species) comprising at least one pyridyldithiol group having a core structure

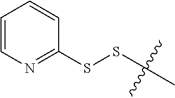 [SMILES: *SSc1ccccn1]

wherein the pyridyldithiol group is capable of forming a covalent bond with a functional group (e.g., a thiol) of a biomolecule. In some embodiments, the pyridyldithiol group can be functionalized, such as on the pyridine ring.

Substrate-Based Platform: A component of an electrochemical biomolecule-functionalized sensor device of the present disclosure that comprises a substrate that is made of a fiber-based material or a substrate made of a solid material that is not fiber-based, wherein the substrate is physically connected to an electrode of the disclosed device embodiments, such as a working electrode as described herein.

Support: A two-dimensional object upon which nanotubes can be grown. In some embodiments, the support is a metal object (e.g., a metal foil) upon which nanotubes can be grown.

II. Device Embodiments

Disclosed herein is an electrochemical biomolecule-functionalized sensor device for use in selectively detecting the presence of particular biological indicators, such as antigens (e.g., proteins, peptides, polysaccharides, and/or combinations thereof), bacteria, viruses, or cellular components thereof, in order to diagnose a disease or condition involving such biological indicators. In particular embodiments, the electrochemical biomolecule-functionalized sensor device is designed to detect antigens associated with infectious diseases, such as COVID-19, or other diseases, such as pneumonia, HIV, malaria, Ebola, MERS COV-2, influenza, hepatitis C, stress-associated hormone-based diseases (Cushing's syndrome), and the like. The disclosed electrochemical biomolecule-functionalized sensor device can be used as a single-use device or as a multi-use device. In some embodiments, the electrochemical biomolecule-functionalized sensor device embodiments disclosed herein can be made with low-cost materials and can be fabricated efficiently and thus avoid the complex fabrication methods and high manufacturing costs associated with current electrochemical sensor devices. Device embodiments disclosed herein are fast, user friendly, inexpensive, and accurate and sensitive.

In particular embodiments, the electrochemical biomolecule-functionalized sensor device comprises a working electrode. In additional embodiments, the electrochemical biomolecule-functionalized sensor device comprises a substrate-based platform that comprises a substrate associated with the working electrode. The working electrode comprises a plurality of functionalized nanotubes. The functionalized nanotubes comprise nanotubes that have been functionalized with a coating comprising a surface binding agent and a biomolecule. In some embodiments, functionalization of the nanotubes can occur either by covering a surface of the plurality of nanotubes with the coating comprising the surface binding agent and the biomolecule, or by covering a surface of one or more individual nanotubes of the plurality with the coating.

The nanotubes that are functionalized with the coating comprising the surface binding agent and the biomolecule are synthesized to have an average length ranging from 1 μm to 4 μm, such as 2 μm to 4μm, or 3 μm to 4 μm. In particular embodiments, the nanotubes have an average length of greater than 3μm. In particular embodiments, nanotubes having these lengths provide an advantage over shorter nanotubes having lengths below 3 um as the longer nanotubes exhibit increased surface area and thus can promote more chemical reactions between biological indicators (e.g., antigens and other such biological indicators disclosed herein) and a biomolecule associated with the nanotubes.

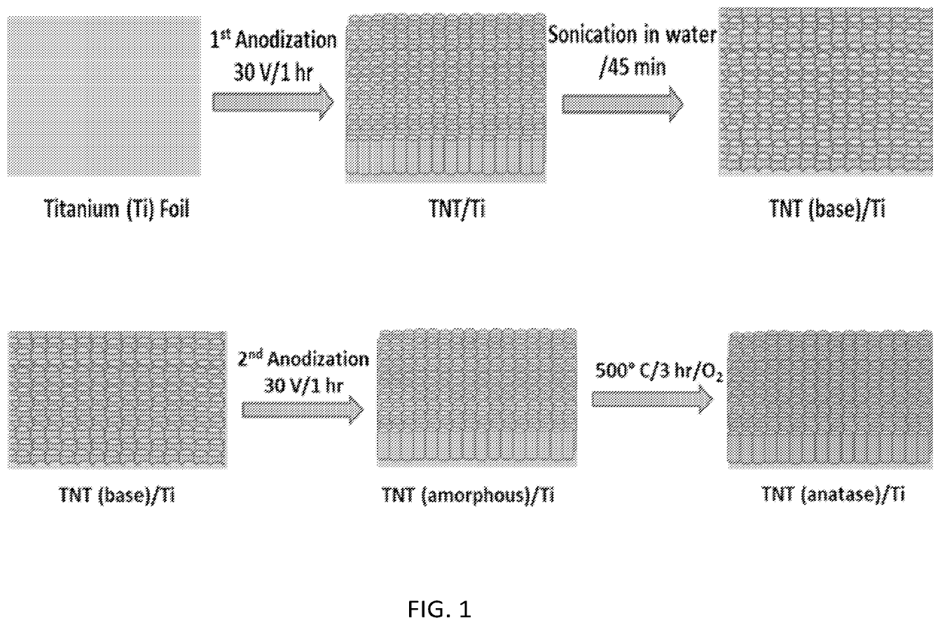

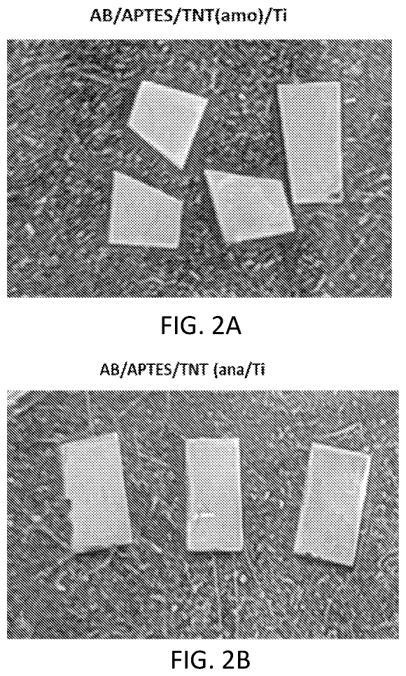

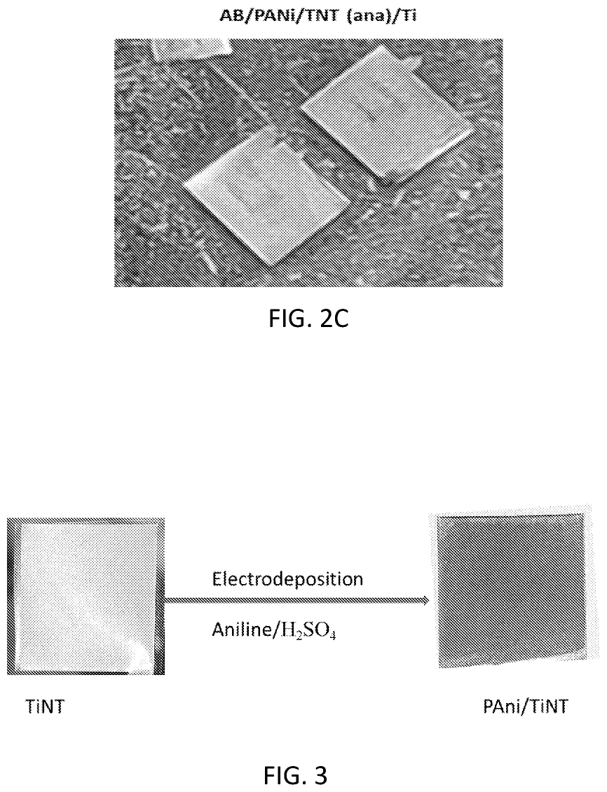

The nanotubes are made of one or more metal oxides. The metal oxide can be a transition metal oxide or a main-group metal oxide. Transition metal oxides can include, but are not limited to, a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof. Main-group metal oxides can include, but are not limited to an aluminum oxide, a silicon oxide, a gallium oxide, an indium oxide, a tin oxide, a lead oxide, or combinations thereof. In representative embodiments, the nanotubes comprise a titanium oxide, such as titanium dioxide (as referred to herein as titania or TiO₂); a tantalum oxide, such as TaO₂, Ta₂O₅, or a combination thereof; a tin oxide (e.g., SnO₂); a zinc oxide (e.g., ZnO); or a combination thereof. In some embodiments, the metal oxide may exist in amorphous form, anatase form, or a combination thereof. In some embodiments, the plurality of nanotubes can comprise nanotubes that are made of an amorphous metal oxide, nanotubes that are made of an anatase form of the metal oxide, or a combination of any such nanotubes. An exemplary illustration of a nanotube array (or plurality of nanotubes) is illustrated in FIG. 1 and FIGS. 2A-2C show images of nanotube arrays that have been coated with a surface binding agent and a biomolecule, wherein the nanotubes are amorphous (FIG. 2A) or in anatase form (FIGS. 2B and 2C).

In particular embodiments, the nanotubes are functionalized with surface binding agent. The surface binding agent can be a polymeric or non-polymeric material. In some embodiments, the surface binding agent is a material comprising functional groups that facilitate covalently binding the surface binding agent to a biomolecule. In particular embodiments, the surface binding agent is an amino-containing compound, a carboxylic acid-containing compound, a maleimide-containing compound, a haloacetyl-containing compound (e.g., an iodoacetyl-containing compound), a pyridyldithiol-containing compound, or a compound containing other functional groups capable of conjugating with functional groups of a biomolecule. The surface binding agent can be electroactive. In some embodiments, the surface binding agent component is a polymeric material comprising amino groups, such as a polyaniline. In some such embodiments, the polyaniline is electroactive. The polyaniline can have an average Mw ranging from 1,000to 100,000, such as 5,000 to 75,000, or 5,000 to 65,000, or 5,000 to 50,000, or 5,000 to 20,000. In particular embodiments, the surface binding agent is an electroactive polyaniline polymer, such as a polyaniline (or “PANi”). In such embodiments, the polyaniline can be (but is not limited to) emeraldine base polyaniline, emeraldine salt polyaniline, leucoemeraldine base polyaniline, and the like. In some embodiments, the surface binding agent can be a non-polymeric material comprising any of the functional groups described above. In particular embodiments, the non-polymeric material is an amino-silane. In such embodiments, the amino-silane can be an aminoalkyl trialkoxy silane, such as aminopropyl triethoxysilane (or “APTES”), 3-aminopropyldimethylethoxysilane (or “APDMES”), 3-aminopropyltrimethoxysilane (or “APTMS”), propyldimethylmethoxysilane (or “PDMMS”), or N-(6-aminohexyl) aminomethyltriethoxysilane (or “AHAMTES”). In some independent embodiments, the coating may further comprise reagents used to facilitate forming the coating, such as reagents described herein that facilitate coupling the surface binding agent to the biomolecule (e.g., NHS, EDC, and other coupling reagents described herein). In some embodiments, a combination of different surface binding agents can be used such that the coating comprises a single layer that is made up of a mixture of the different surface binding agents and/or such that the coating comprises a first layer of a first surface binding agent formed on the nanotubes, followed by a second layer of a second different surface binding agent formed on the first layer comprising the first surface binding agent.

The biomolecule can be a biological compound that is capable of binding with a biological indicator present in a sample. In some embodiments, the biomolecule is capable of binding an epitope of an antigen present in a biological sample that is applied to the electrochemical biomolecule-functionalized sensor device. In particular embodiments, the biomolecule can be an antibody, a polynucleotide, a protein, a peptide, or a combination thereof. In representative embodiments, the biomolecule is an antibody or a C-reactive protein. In some embodiments, a plurality of biomolecules (e.g., antibodies) can be used, wherein the plurality comprises the same biomolecule species or a combination of different biomolecule species. In some embodiments, the plurality can comprise the same type of biomolecule (e.g., antibody, polynucleotide, or protein), which in turn can comprise the same or different species of any such biomolecule; or the plurality can comprise a combination of different types of biomolecules, wherein each type can comprise the same or different species of that particular biomolecule. In particular embodiments, the biomolecule is an antibody that specifically binds antigens associated with COVID-19, such as an anti-spike RBD antibody. In such embodiments, the antibody can be one that specifically binds the SARS-COV-2 spike protein (or “s-RBD”) associated with COVID-19. In some additional embodiments, the antibody can be one that specifically binds the nuclear protein of SARS-CoV0-2 virus, or an antigen of other viral diseases.

In particular embodiments, the nanotubes are covered with the coating comprising the surface binding agent and the biomolecule such that the nanotubes are physically associated (e.g., in direct contact) with the coating and/or are chemically bound to the coating. In particular embodiments, the nanotubes are physically associated with the surface binding agent such that a physical layer of the surface binding agent is deposited on the nanotubes, but chemical bonding need not occur. In some embodiments, the nanotubes are chemically bound to the surface binding agent through covalent bonds, ionic bonds, and/or electrostatic bonds. In particular embodiments, there is a chemical interaction between the surface binding agent and the nanotubes, which can occur through a coordination bond between a functional group of the surface binding agent (e.g., a nitrogen atom-containing functional group, such as an amine) and the nanotubes; or through a chemisorption interaction involving covalent Si—O—Ti bonding between an amino-silane surface bonding agent and the nanotubes. FIG. 3 shows photographs of a nanotube-functionalized substrate before (left-most image) and after (right-most image) application of the surface binding agent. The surface binding agent, in turn, is chemically bound to the biomolecule, typically through one or more chemical bonds, such as a covalent, ionic, or electrostatic bond. In particular embodiments, each biomolecule included in the coating is covalently bound to one or more functional groups of the surface binding agent. In exemplary embodiments, the surface binding agent is a polyaniline or amino-silane component comprising amino groups that covalently bind with carboxylic acid groups of the biomolecule, such as carboxylic acid groups of an antibody, a polynucleotide, or a protein. In yet other embodiments, the surface binding agent can be a compound or material comprising carboxylic acid groups that can bind with available amino groups of the biomolecule, such as amino groups of an antibody, a polynucleotide, or a protein. In yet some additional embodiments, the surface binding agent can be a compound or material comprising functional groups that can bind with thiols present on an antibody, a polynucleotide, or a protein. In such embodiments, the surface binding agent can comprise a maleimide-containing compound, a haloacetyl-containing compound (e.g., an iodoacetyl-containing compound), a pyridyldithiol-containing compound, or a combination thereof.

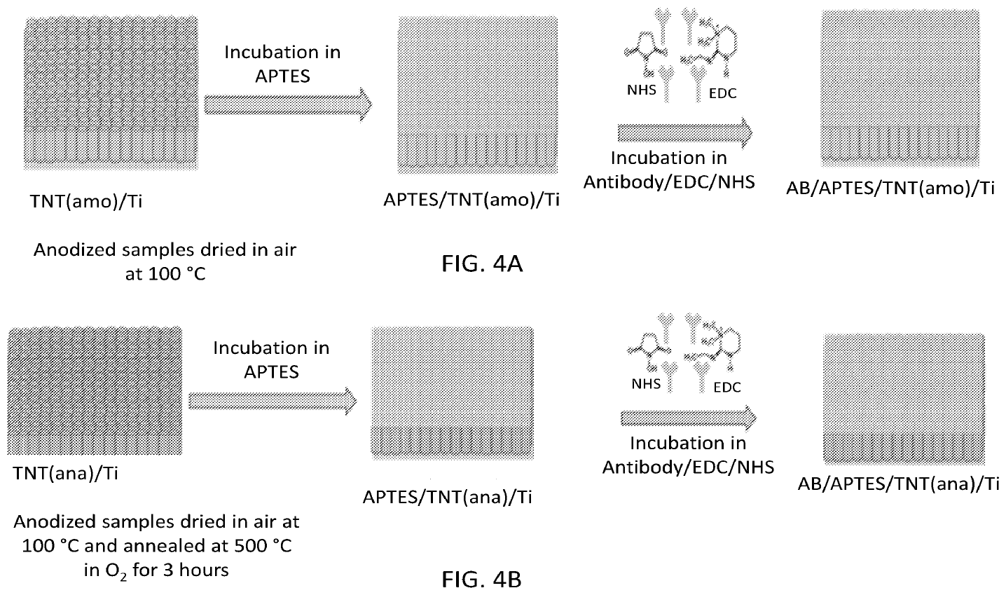

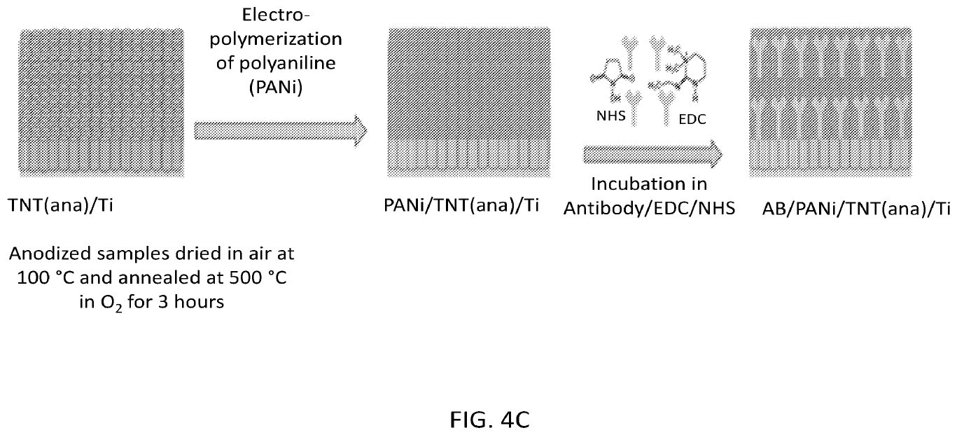

The coating comprising the surface binding agent and the biomolecule typically is a uniform, thin layer formed on the nanotubes of the working electrode. In particular embodiments, the thin layer has a thickness ranging from 0.5 Å to 400 Å, such as 0.5 Å to 1000 Å, or 0.5 Å to 500 Å. The layer of the coating is uniform such that the thickness of the film remains substantially constant across the surface area comprising the coating. In some embodiments, a single layer, or “monolayer,” of the coating is applied such that any biomolecules of the coating are capable of physically interacting with biological indicators (e.g., antigens and other such biological indicators disclosed herein) present in a sample applied to the electrochemical biomolecule-functionalized sensor device. In particular embodiments, the biomolecules are antibodies that are directionally oriented such that antigen-binding regions of the antibody extend vertically upwards, away from the nanotubes. An exemplary illustration of this type of orientation is illustrated in FIGS. 4A-4C. In particular embodiments, the coating covers the entire surface area of the top of the nanotubes which are not attached to the substrate of the working electrode. In some such embodiments, the coating can cover any exposed surface of the nanotubes.

In some embodiments, the substrate component that can be physically attached to the working electrode to form the substrate-based platform can be made of a fiber-based material or the substrate can be made of a solid material that is not fiber-based. In some embodiments, the fiber-based material can comprise a cellulosic fiber material capable of wicking, such as a paper-type material, or a synthetic fiber material. In some such embodiments, the cellulosic fiber material can be obtained from any source, such as from natural materials (e.g., wood, hemp, linen, cotton, or the like). In some embodiments, the cellulosic fiber material can be a cellulose paper or other porous paper material. In some embodiments, the synthetic fiber materials can include, but are not limited to, a polyester fiber material, an acrylic fiber material, or the like. In yet other embodiments, the substrate-based platform can further comprise a film material. In some such embodiments, the film material can comprise a thermally conductive polyimide film (e.g., a Kapton® film sold by DuPont), a porous paper-based material, or other transparent plastic and/or polymeric materials. In some embodiments, the film material can be used as a support material for other components of the substrate-based platform, or it can be used to adhere such components to the substrate component of the substrate-based platform. In some other embodiments, the substrate can comprise a plastic material (e.g., biaxially oriented polypropylene (BOPP) or high-density polyethylene (HDPE)), a metal material (e.g., non-conductive metal materials and/or conductive metal materials, such as a metal foil); a glass material; or any combination thereof.

In particular embodiments, the substrate-based platform further comprises a plurality of electrodes attached to, or printed onto, the substrate, in addition to the working electrode. These other electrodes can include a counter electrode, a reference electrode, or a combination thereof. The reference electrode can be any suitable reference electrode, such as an Ag/AgCI reference electrode. The counter electrode can be any suitable counter electrode, such as a titanium electrode

The electrochemical biomolecule-functionalized sensor device can further comprise other components in addition to the working electrode and/or the substrate-based platform, such as a power source, a potentiostat, a housing, a sample introduction inlet or region, connective components (e.g., wires, clamps, adhesives, or the like), control mechanisms, and/or electronic displays. In particular embodiments, the electrochemical biomolecule-functionalized sensor device comprises a power source that is integrated with the electrochemical biomolecule-functionalized sensor device or that is separate from the electrochemical biomolecule-functionalized sensor device. For example, in embodiments comprising an integrated power source, it can be a built-in rechargeable, disposable, or replaceable battery. In embodiments using an external power source, it can be connected to the electrochemical biomolecule-functionalized sensor device through wires or can be paired with the electrochemical biomolecule-functionalized sensor device using other means. The power source can be a direct current or alternating current power source. In particular embodiments, the power source is a battery and in some embodiments can be a battery of a cellular device or other portable electronic device. The power source is configured to supply sufficient power so as to generate a voltage (such as a bias voltage) suitable for actuating the electrochemical biomolecule-functionalized sensor device. In some embodiments, the power generated from the power source can be tuned to provide a particular voltage that is selected depending on the type of biological indicators (e.g., antigens and other such biological indicators disclosed herein) to be detected using the device. In some embodiments, a voltage suitable for detecting antigens associated with COVID-19 can include a voltage ranging from −0.1 V to −1.2 V, such as −0.2 V to −1.2 V, or −0.3 V to −1 V, or −0.5 V to −1 V, or −0.6 V to −1V, or −0.3 V to −0.6 V. In some embodiments, the voltage can range from −0.1 V to −0.7V, such as −0.45 V to −0.35 V, or −0.5 V to −0.8 V. In particular embodiments, the electrochemical biomolecule-functionalized sensor device further comprises a potentiostat that is integrated with the electrochemical biomolecule-functionalized sensor device and that can facilitate measuring any current (or change in current) generated by the electrochemical biomolecule-functionalized sensor device during use.

In some embodiments, the electrochemical biomolecule-functionalized sensor device is reusable. In some such embodiments, the electrochemical biomolecule-functionalized sensor device can be treated so as to remove the coating comprising the surface binding agent and the biomolecule. In some additional such embodiments, the electrochemical biomolecule-functionalized sensor device can be exposed to UV light to passivate the coating.

The electrochemical biomolecule-functionalized sensor device also can include integrated controls, or it can be configured to be controlled by a personal computer, laptop, smart phone, or other smart electronic devices. The electrochemical biomolecule-functionalized sensor device can further comprise an electronic display that can be used to view results from the electrochemical biomolecule-functionalized sensor device. In some embodiments, the electrochemical biomolecule-functionalized sensor device can display a graphical representation of the current signal from the electrochemical biomolecule-functionalized sensor device, or it can provide a verbal cue to indicate whether or not a biological indicator (e.g., antigens and other such biological indicators disclosed herein) is present (e.g., “present” or “not present”; “positive” or “negative”; “yes” or “no”; or the like); an audio cue (e.g., a beep or other alarm indicating that a biomarker is present); or any combination thereof. In some embodiments, the electrochemical biomolecule-functionalized sensor device can be encased within a housing that may contain all components or only certain components of the electrochemical biomolecule-functionalized sensor device. In particular embodiments, the housing may contain the substrate-based platform, the potentiostat, and the power source, if the power source is integrated. In embodiments comprising a housing, an opening is provided so as to facilitate delivery of the sample to the substrate and/or the working electrode comprising the plurality of nanotubes.

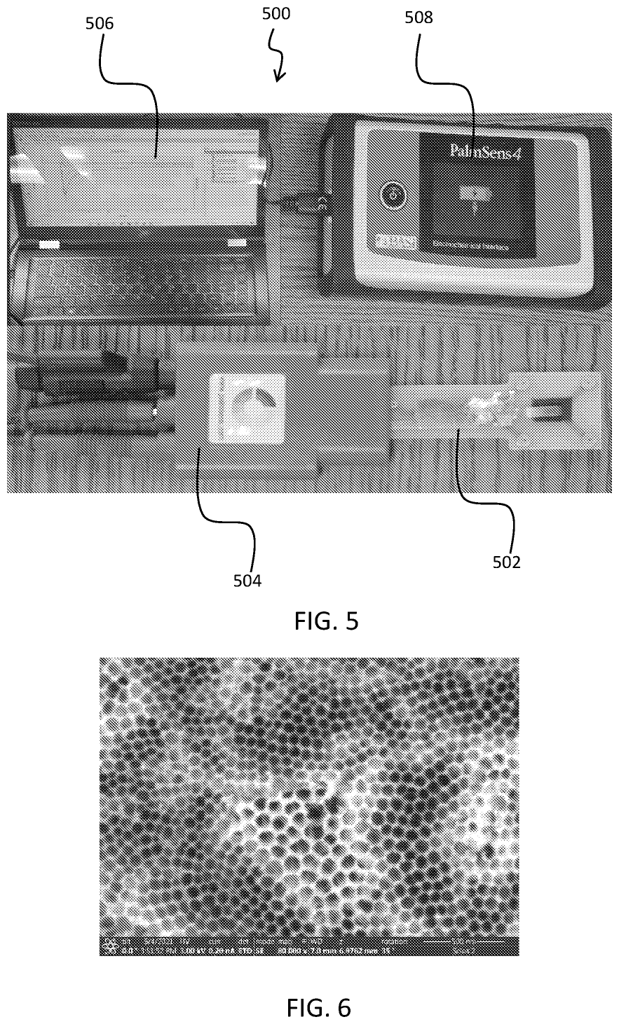

Components and configurations used in an exemplary electrochemical biomolecule-functionalized sensor device embodiment are shown in FIG. 5. FIG. 5 provides a photographic image of an exemplary device set-up 500, comprising substrate-based platform 502, screen-printed-electrode connector 504, read-out display component 506, and potentiostat 508.

III. Method Embodiments

Disclosed herein are embodiments of a method of making and using the electrochemical biomolecule-functionalized sensor device embodiments disclosed herein. In particular, method embodiments for making the functionalized nanotubes associated with a coating comprising a surface binding agent and a biomolecule are described, along with methods of using the electrochemical biomolecule-functionalized sensor device. As discussed herein, “associated with a coating” means that the nanotubes can be physically associated with the coating such that the two components are in direct contact, or the nanotubes can be chemically bound to the coating.

The nanotubes of the electrochemical biomolecule-functionalized sensor device can be made using an anodization method in combination with one or more deposition methods (e.g., electrodeposition). In some embodiments, the nanotubes themselves are made using a double anodization method. In such embodiments, two separate anodization steps can be used in combination with one or more annealing steps to provide nanotubes comprising the metal oxide material. The nanotubes made with this double anodization method exhibit increased lengths relative to nanotubes made with a single anodization step. In particular embodiments, the method comprises performing a first anodization step and a second anodization step. In some embodiments, the first and/or second anodization steps can comprise exposing a metal oxide precursor component (or intermediate product formed therefrom, such as that obtained after a first anodization step), to an electrochemical bath comprising an anodizing solution. In some embodiments, the same electrochemical bath/anodizing solution can be used for each step. The electrochemical bath is subjected to a suitable voltage. In each of the first and second anodization steps, independently, the voltage can range from 10 V to 30 V, such as 20 V to 45 V, or 25 V to 30V. In some embodiments, the anodizing solution can comprise an electrolyte selected from a fluoride-containing salt, a chloride-containing salt, a chromium-containing salt, a bromide-containing salt, a perchlorate-containing salt, water, and ethylene glycol. In particular embodiments, the anodizing solution comprises an electrolyte selected from ammonium fluoride, sodium fluoride, hydrofluoric acid, or a combination thereof; water; and ethylene glycol. The metal oxide precursor component (or intermediate product formed therefrom, such as that obtained after a first anodization step) can be held in the electrochemical bath at the applied voltage for a period of time sufficient to form nanotubes. In each of the first and second anodization steps, independently, the amount of time in the electrochemical bath can range from 25 minutes to 240 minutes, such as 20 minutes to 90 minutes, or 20 minutes to 45 minutes, or 30 minutes to 60 minutes.

In some embodiments, the method can further comprise performing one or more washing steps, one or more annealing steps, or a combination thereof. In some embodiments, the method comprises one or more washing steps, which can comprise rinsing the intermediate product formed after a first anodization step (or a final nanotube array formed from the second anodization step) with a solvent, such as an alcohol (e.g., ethanol), and then sonicating the intermediate or final product in water. In some embodiments, the intermediate product and/or the final product are sonicated for an hour or less. In additional embodiments, the method can further comprise one or more annealing steps. In any such embodiments, the annealing step can comprise heating the intermediate product and/or a final nanotube array formed from the second anodization step in the presence of oxygen at a temperature ranging from 200° C. to 600° C., such as 300° C. to 575° C., or 400° C. to 550° C., or 450° C. to 500° C. for 1 to 10 hours, such as 1, 2, 3, 4, 5, 6, 7, 8, 9, or 10 hours. The annealing step can be performed after a first anodization step, a second anodization step, or both. In some embodiments, the annealing step can be used to convert amorphous forms of the metal oxide nanotubes to an anatase form of the metal oxide nanotubes. In some embodiments, the method can comprise additional anodization steps, such as a third, fourth, or fifth anodization steps, which can comprise the processes described above for the first and/or second anodization steps.

In some embodiments, the method can further comprise depositing a surface binding agent on the nanotubes by exposing the nanotubes to a solution comprising the surface binding agent either after a second anodization step or after an annealing step performed after the second anodization step. In some embodiments, the solution comprising the surface binding agent can comprise a single surface binding agent moiety or a mixture of different surface binding agents. Representative method embodiments use a solution comprising a single surface binding agent, such as a polyaniline material or an amino-silane compound. In some independent embodiments, however, a mixture of polyaniline and amino-silane can be used as the surface binding agent. The solution comprising the surface binding agent can be applied using a suitable technique for depositing a layer of the surface binding agent on exposed surfaces of the nanotubes. In some embodiments, the solution can be deposited by an immersion technique, an electrochemical deposition technique, a drop deposition technique (e.g., drop casting), a dip-coating technique, a printing technique, a chemical vapor deposition technique, a laser deposition technique (typically a low-temperature laser deposition technique), a plasma deposition technique, or the like. If the surface binding agent comprises a polyaniline material, the method typically comprises using an electrochemical deposition method or a drop deposition method. Embodiments using electrochemical deposition can comprise exposing the nanotubes to an electrolyte solution comprising the polyaniline and an acid (e.g., sulfuric acid) at a constant potential (e.g., 1.5 V for 400 seconds). In some such embodiments, the nanotubes comprise the metal oxide in anatase form. In other embodiments using different surface binding agents (e.g., an amino-silane or other surface binding agents disclosed herein), the method can comprise using any of the above-mentioned deposition techniques, with particular embodiments using an immersion technique, wherein the nanotubes are immersed in a solution comprising the surface binding agent and incubated for a suitable amount of time to form a layer of the surface binding agent on exposed surfaces of the nanotubes. In some embodiments, this time period can range from 1 hour to 12 hours, such as 1 hour to 8 hours, or 1 hour to 6 hours. After the nanotubes have been modified with a layer of the surface binding agent, the nanotubes can be treated with an acid and/or washing solution. In embodiments where a polyaniline is used as the surface binding agent, the nanotubes can be treated with sulfuric acid after being modified with the polyaniline, followed by washing with a solvent (e.g., an alcohol, such as ethanol; or other organic solvents, like acetone). A similar washing step can be used to wash other embodiments using different surface binding agents, with particular embodiments using acetone. The method can further comprise allowing the functionalized nanotubes to dry, such as by drying them under heat (e.g., exposing the nanotubes to temperatures above ambient temperature to 60° C.) or air drying by leaving them exposed to air or a flowing inert gas. Drying can be conducted in vacuo in some embodiments.

In particular embodiments, the method further comprises exposing the nanotubes functionalized with the surface binding agent to the biomolecule to form the coating on the nanotubes. The biomolecule typically is provided as a solution. In some embodiments, the solution comprising the biomolecule can further comprise one or more coupling reagents that facilitate binding the biomolecule to the surface binding agent. The coupling reagents can be selected from reagents suitable for the type of surface binding agent and biomolecule being used to functionalize the nanotubes. In some embodiments using a surface binding agent and biomolecule pairing wherein the surface binding agent comprises an amino group and the biomolecule comprises a carboxylic acid group (or vice versa), the coupling reagents can be reagents that facilitate forming an amide bond between these two groups. Exemplary such reagents can include, but are not limited to, (1-ethyl-3-(3-dimethylamino) propyl carbodiimide (or “EDC”), N-hydroxysuccinimide (or “NHS”), N-hydroxysulfosuccinimide (or “sulfo-NHS”), 2-(7-aza-1H-benzotriazol-1-yl)-N,N,N′,N′-tetramethylaminium hexafluorophosphate (or “HATU”), 2-(1H-benzotriazol-1-yl)-N, N,N′,N′-hexafluorophosphate (or “HBTU”), 2-(6-chloro-1H-benzotriazol-1-yl)-N, N, N′, N′-tetramethylaminium 15 hexafluorophosphate (or “HCTU”), 1-hydroxybenzotriazole (or “HOBt”), dicyclohexylcarbodiimide (or “DCC”), diisopropylcarbodiimide (or “DIC”), benzotriazol-1-yloxy-tris (dimethylamino)-phosphonium hexafluorophosphate (or “BOP”), benzotriazol-1-yloxy-tripyrrolidino-phosphonium hexafluorophosphate (or “PyBOP”), bromo-tripyrrolidino-phosphonium hexafluorophosphate (or “PyBroP”), or any combination thereof. In embodiments wherein the surface binding agent comprises a thiol-reactive group, such as maleimide group, a haloacetyl group, or a pyridyldithiol group, and the biomolecule comprises a thiol group (or a group capable of providing a thiol group, such as a disulfide moiety), the solution comprising the biomolecule can comprise a buffer that facilitates maintaining a desired pH suitable for forming sulfur-carbon or disulfide bonds between the surface binding agent and the biomolecule. In some embodiments, the pH can range from 4 to 8 (such as 5 to 7.5, or 6 to 7.5, or 6.5 to 7.5) so as to promote forming a carbon-sulfur bond between a maleimide-containing surface binding agent and a thiol-containing biomolecule or forming a disulfide bond between a pyridyldithiol group and a thiol-containing biomolecule. In other embodiments, the pH can be maintained above 7.5 so as to facilitate forming a carbon-sulfur bond between a haloacetyl-containing surface binding agent and a thiol-containing biomolecule. A person of ordinary skill in the art with the benefit of the present disclosure would recognize suitable amounts of the coupling reagents that can be used to facilitate binding the biomolecule to the surface binding agent. In some embodiments using EDS and NHS (or other similar coupling reagent combinations), the EDC: NHS ratio can range from 1:2 to 1:10. In some representative embodiments using EDC and NHS as coupling reagents, a molar ratio of 1:2 EDC to NHS is used.

The biomolecule can be deposited on the nanotubes that have been modified by the surface binding agent using a suitable deposition technique. In some embodiments, a solution comprising the biomolecule can be added on top of the layer of the surface binding agent deposited on the nanotubes using an immersion technique, a drop deposition technique (e.g., drop casting), a dip-coating technique, a printing technique, a laser deposition technique (typically a low-temperature laser deposition technique), a plasma deposition technique, or the like. In particular embodiments, a solution comprising the biomolecule (and, optionally, the coupling reagent(s)) is drop cast on to the layer of the surface binding agent. In some embodiments, a single layer of the biomolecule can be deposited on the nanotubes such that the same antibody molecules cover the entire surface provided by the nanotubes. In yet other embodiments, the surface of the nanotubes can be patterned with different types of antibodies. For example, patterned rows can be formed on the nanotubes such that one patterned layer comprises molecules of one particular antibody species and one or more other patterned layers comprise molecules of a different antibody species.

In some embodiments, the method can further comprise adding a blocking agent to the substrate-based platform after the biomolecule has been bound to the surface binding agent. The blocking agent can facilitate blocking any regions of the nanotubes that have not been functionalized with the surface binding agent, the antibody, or both such components. In particular embodiments, the blocking agent blocks areas on the nanotubes that have not be associated with an antibody. Blocking such regions of the substrate-based platform can help prevent non-specific adsorption of any biological indicators (e.g., antigens and other such biological indicators disclosed herein) being detected. In particular embodiments, the blocking agent can be bovine serum albumin, hexylamine, hydroxylamine, ethanolamine, 1,3-propyldiamine, or a combination thereof.

In particular embodiments, the electrochemical biomolecule-functionalized sensor device can be constructed to be compatible in methods for detecting particular biological indicators (e.g., antigens and other such biological indicators disclosed herein) in order to diagnose a particular disease or condition. For example, a device embodiment can be constructed to comprise substrate-based platform having a working electrode as described herein. Additional electrodes can be paired with the substrate-based platform, along with other components described herein for the device. In particular embodiments, the electrochemical biomolecule-functionalized sensor device is constructed for use with fluid samples, particularly liquid samples. The sample can be a biological sample obtained from a subject. In particular embodiments, the biological sample can be condensed breath, breath vapor, saliva, mucous, blood, or other forms of biological samples that can be obtained and provided in liquid form. In embodiments where a condensed breath sample is used, the sample can be collected by having a subject breath into a breath bag or other breath-capturing device and then obtaining condensate therefrom. In such embodiments, rather than testing a subject's breath using gas analysis of the breath with the electrochemical biomolecule-functionalized sensor device, liquid-based analysis can be used. In representative embodiments, the sample is a breath, saliva, or nasal mucous (or nasopharyngeal) sample obtained from a subject. In some embodiments, the sample can be dissolved or diluted with water prior to analysis using the electrochemical biomolecule-functionalized sensor device.

In particular embodiments, the electrochemical biomolecule-functionalized sensor device can be used to detect the presence of biological indicators (e.g., antigens and other such biological indicators disclosed herein) to thereby diagnose a subject that has, or that may develop, a condition or disease associated with any such biological indicator. In some embodiments, the biological indicator is an antigen. In some such embodiments, the antigen is the s-RBD protein. In some embodiments, the disease or condition can be, but is not limited to, COVID-19, and other diseases, such as pneumonia, HIV, malaria, Ebola, MERS COV-2, influenza, hepatitis C, stress-associated hormone-based diseases (e.g., Cushing's syndrome), and the like. The coating comprising the surface binding agent and biomolecule that is positioned on the nanotubes of the working electrode facilitates the ability to bind the biological indicator (e.g., antigens, such as proteins, peptides, polysaccharides, and/or combinations thereof; bacteria; viruses; or cellular components thereof), which can then result in the sensor exhibiting an electrochemical change that can be detected.

Particular method embodiments of using the electrochemical biomolecule-functionalized sensor device to detect, identify, and/or quantify an analyte present in a sample are disclosed. In some embodiments, the method comprises applying a voltage to the electrochemical biomolecule-functionalized sensor device, measuring a current produced by the electrochemical biomolecule-functionalized sensor device, exposing the electrochemical biomolecule-functionalized sensor device to a sample, sensing a change in current produced by the electrochemical biomolecule-functionalized sensor device, and measuring the change in current. In yet additional embodiments, the method can further comprise diagnosing a subject having, or is at risk of developing, a condition or disease using results produced by the electrochemical biomolecule-functionalized sensor device.

A voltage can be applied to the electrochemical biomolecule-functionalized sensor device using a power source connected to the electrochemical biomolecule-functionalized sensor device, which may be a separate component or integrated with the electrochemical biomolecule-functionalized sensor device. The applied voltage can be as described herein, with particular embodiments having an applied voltage ranging from −0.1 V to −0.7 V, such as −0.45 V to −0.35 V, or −0.5 V to −0.7 V. In some embodiments, different voltages can be applied, such as when different biomolecules have been included in the coating to provide the ability to detect multiple different biological indicators with the same device. Current produced by the electrochemical biomolecule-functionalized sensor device can be measured using any suitable means for measuring an electrical current. In some embodiments, cyclic voltammetry is used. In yet other embodiments, amperometry is used. In some embodiments, cyclic voltammetry and amperometry can be used. In particular embodiments, the current is measured to obtain a baseline current that is emitted by the electrochemical biomolecule-functionalized sensor device with or without being exposed to a sample. The current also may be measured to determine the presence of, the identity of, or the amount of, a biological indicator present in a sample. In particular embodiments, the current produced by the electrochemical biomolecule-functionalized sensor device is measured using the potentiostat and displayed on a computer or other display device electrically connected to the potentiostat. In particular embodiments, rapid detection of biological indicators is possible with the disclosed electrochemical biomolecule-functionalized sensor device. In some embodiments, detection can be accomplished in as little as 30 seconds or less, with some embodiments taking 20 seconds or less, and even five seconds or less.

The electrochemical biomolecule-functionalized sensor device can be exposed to the sample by physically associating the sample with the electrochemical biomolecule-functionalized sensor device in any suitable manner so as to facilitate chemical, electrical, and/or physical contact between the sample and the biomolecules of the coating formed on the functionalized nanotubes of the working electrode. The electrochemical biomolecule-functionalized sensor device can comprise a sample introduction region and/or a sample introduction inlet that is used to apply the sample to the electrochemical biomolecule-functionalized sensor device for use. In some embodiments, the electrochemical biomolecule-functionalized sensor device is configured in a manner such that the sample can be introduced into the electrochemical biomolecule-functionalized sensor device so as to be in direct contact with the working electrode. In yet other embodiments, the electrochemical biomolecule-functionalized sensor device is configured in a manner such that the sample comes into contact with the working electrode through wicking or capillary action provided by the cellulosic fiber material or the synthetic fiber material of the substrate-based platform. In particular embodiments, the electrochemical biomolecule-functionalized sensor device is exposed to the sample using a swab to administer a liquid sample to the electrochemical biomolecule-functionalized sensor device. In such embodiments, the swab can be used to directly swab a sample from the subject by swabbing the subject's mouth or nose and then contacting the electrochemical biomolecule-functionalized sensor device with the swab. In other embodiments, the swab can be used to swab a liquid sample that has been obtained from a subject and subsequently stored in a container. The amount of sample needed for use with the device is minimal and in some embodiments, the amount can be less than 1000 nanograms/liter, such as 0.1 nanogram/liter to 1000 nanograms/liter, or 1 nanogram/liter to 1000 nanograms/liter, or 4 nanograms/liter to 1000 nanograms/liter, or 4 nanograms/liter to 100 nanograms/liter, or 1 nanogram/liter to 50 nanograms/liter

After the electrochemical biomolecule-functionalized sensor device has been exposed to a sample, a change in current produced by the electrochemical biomolecule-functionalized sensor device can be sensed and/or measured. In particular embodiments, the change in current can comprise an increase of current from the current observed or measured prior to sample addition. In such embodiments, the term “increase” means that the current becomes more negative. In some embodiments, the change in current can be measured. In such embodiments, measuring the change in current produced by the electrochemical biomolecule-functionalized sensor device after exposing it to the sample can indicate that the biomolecule has bound a biological indicator present in the sample (e.g., an antibody has bound an antigen). The electrochemical biomolecule-functionalized sensor device can be used for selective analyte detection as it can provide a signal upon binding of the desired biological indicator to the biomolecule, whereas other compounds that might be present in the sample do not bind and thus do not provide any signal. In some embodiments, measuring the change in current produced by the electrochemical biomolecule-functionalized sensor device after exposing it to the sample can facilitate identifying the analyte in terms of its chemical identity. For example, a particular analyte may provide a particular current change and thus the value of the current change can be used to characterize the analyte. In yet additional embodiments, measuring the change in current produced by the electrochemical biomolecule-functionalized sensor device after exposing it to the sample can provide an indication as to how much of the analyte is present in the sample. In some embodiments, the antigen/antibody interaction can be used to quantify the amount of antigen present in a sample using a suitable technique, such as SEM analysis and applying standards and/or algorithms known to those of ordinary skill in the art with the benefit of the present disclosure.

IV. Overview of Several Embodiments

Disclosed herein are embodiments of an electrode, comprising a plurality of functionalized nanotubes on a support, wherein the functionalized nanotubes comprise metal oxide-based nanotubes that are associated with a coating comprising a surface binding agent and biomolecule.

In any or all embodiments, the metal oxide-based nanotubes of the plurality of functionalized nanotubes have an average length greater than 3 mm.

In any or all embodiments, the metal oxide-based nanotubes comprise a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof.

In any or all embodiments, the metal oxide-based nanotubes are TiO₂ nanotubes that exist in anatase form, amorphous form, or a combination thereof.

In any or all embodiments, the surface binding agent is an amino-containing compound, a carboxylic acid-containing compound, a maleimide-containing compound, a haloacetyl-containing compound, a pyridyldithiol-containing compound, or a combination thereof.

In any or all embodiments, the amine-containing compound is a polyaniline polymer or a salt thereof; an amino-silane compound; or a combination thereof.

In any or all embodiments, the polyaniline polymer has an average M_{w} ranging from 1,000 to 100,000.

In any or all embodiments, the amino-silane compound is aminopropyl triethoxysilane, 3-aminopropyldimethylethoxysilane, 3-aminopropyltrimethoxysilane, propyldimethylmethoxysilane, or N-(6-aminohexyl) aminomethyltriethoxysilane.

In any or all embodiments, the biomolecule is capable of specifically binding an antigen.

In any or all embodiments, biomolecule is an antibody.

In any or all embodiments, the antibody specifically binds a COVID-19 antigen.

In any or all embodiments, the antibody is covalently bound to the surface binding agent such that antigen-binding regions of the antibody extend vertically upwards and wherein the surface binding agent is coated on top of the functionalized nanotubes.

In any or all embodiments, the electrode further comprises a blocking agent that covers regions of the functionalized nanotubes and/or surface binding agent that do not further comprise a biomolecule.

In any or all embodiments, the support is a metal support.

In any or all embodiments, the electrode further comprises a substrate physically attached to the support of the electrode.

In any or all embodiments, the substrate comprises a fiber-based material, a plastic material, a glass material, or a metal material.

Also disclosed herein are embodiments of a substrate-based platform comprising an electrode according to any or all of the above embodiments, and a substrate physically attached to the support of the electrode.

In any or all embodiments, the functionalized nanotubes comprise TiO₂-based nanotubes functionalized with a coating comprising (i) a layer of a surface binding agent selected from a polyaniline polymer, an amino-silane compound, or a combination thereof; and (ii) an antibody.

In any or all embodiments, the antibody is covalently bound to the layer of the surface binding agent and the coating covers the plurality of functionalized nanotubes.

Also disclosed herein are embodiments of an electrochemical biomolecule-functionalized sensor device, comprising: a working electrode component, comprising the electrode of any or all of the above embodiments; or the substrate-based platform of any or all of the above embodiments; a reference electrode; a counter electrode; and a potentiostat.

In any or all embodiments, the sensor further comprises a power source.

In any or all embodiments, the sensor further comprises a sample introduction inlet or region; a housing; or a combination thereof.

Also disclosed herein are embodiments of a method, comprising: applying a voltage to the electrochemical biomolecule-functionalized sensor device according to any or all of the above embodiments; exposing the electrochemical biomolecule-functionalized sensor device to a biological sample; and sensing a change in current produced by the electrochemical biomolecule-functionalized sensor device after being exposed to the biological sample.

In any or all embodiments, exposing the sensor device to the biological sample comprises contacting the working electrode or the substrate-based platform of the sensor device with the biological sample.

In any or all embodiments, the method further comprises measuring the change in current produced by the electrochemical biomolecule-functionalized sensor device.

In any or all embodiments, the change in current produced by the electrochemical biomolecule-functionalized sensor device signifies a binding event between a biological indicator present in the biological sample and biomolecule of the working electrode or the substrate-based platform.

In any or all embodiments, the biological indicator is an antigen, a bacterium, a virus, or a cellular component thereof.

In any or all embodiments, the biological indicator is SARS-COV-2 spike protein and the biomolecule is an antibody.

In any or all embodiments, the method further comprises diagnosing a subject from which the biological sample is obtained, wherein the subject has, or is at risk of developing, a physiological condition or disease.

In any or all embodiments, the disease is COVID-19.

In any or all embodiments, the voltage is applied to the sensor device using a power source.

In any or all embodiments, the power source is integrated in the electrochemical biomolecule-functionalized sensor device or wherein the power source is an external power source.

In any or all embodiments, the biological sample is a saliva sample, a nasal mucous sample, a breath sample, or a combination thereof.

Also disclosed herein are embodiments of a method for making the electrode of any or all of the above embodiments, the method comprising: performing a first anodization of the support to obtain the metal oxide-based nanotubes formed thereon; performing a second anodization of the metal oxide-based nanotubes to increase the length of the metal oxide-based nanotubes; depositing the surface binding agent on the metal oxide-based nanotubes to form a layer of the surface binding agent on surfaces of metal oxide-based nanotubes; and depositing a solution comprising the biomolecule onto the layer of the surface binding agent.

In any or all embodiments, the solution comprising the biomolecule further comprises one or more coupling reagents.

In any or all embodiments, depositing the surface binding agent comprises using an electrochemical deposition technique or a drop deposition technique.

In any or all embodiments, depositing the solution comprising the biomolecule comprises using a drop deposition technique.

In any or all embodiments, the method further comprises depositing a blocking agent after depositing the solution comprising the biomolecule.

V. Examples

### Example 1

In this example, nanotubes comprising TiO2 were prepared. The nanotubes were prepared by anodizing titanium foils (1 square inch) in an electrolytic solution comprising 0.5 wt % NH₄F and 5 vol % H₂O in ethylene glycol under an ultrasonically agitated condition using an ultrasonic bath (100 W, 42 KHZ, Branson 2510R-MT). A two-electrode configuration was used for anodization. A flag-shaped platinum (Pt) electrode served as a cathode. The anodization was carried out by applying a potential of 20 V using a rectifier (Agilent, E3640A) for 30 minutes. The as-anodized TiO2 samples were washed with water and then placed in an oven at 100° C. for 3 hours. The anodization process was then repeated, followed by annealing in oxygen at 500° C. for 2 hours. In particular examples, a sub-zero temperature was maintained during anodization to avoid artifacts on the titanium substrate.

### Example 2

In this example, which is summarized schematically in FIG. 1, a 1.5 sq cm of titanium (Ti) foil was mechanically polished with sandpaper and then ultrasonically cleaned with acetone, ethanol, and deionized (DI) water for 15 minutes. Anodization of the foil was performed in a electrochemical bath comprising 0.5 wt % ammonium fluoride, and 5 vol % H2O in ethylene glycol for 60 minutes at 30 V. The anodized titanium foil was rinsed with ethanol followed by sonication for 30 minutes in water. The foil was placed again in the same electrochemical bath to undergo a second anodization step, under similar anodization parameters as the first anodization step. The double anodized titanium foil having nanotubes on top was then washed with ethanol and DI water with brief sonication. The amorphous titanium-based nanotubes were annealed for 3 hours at 500° C. in the presence of O2 to convert the nanotubes into anatase form. Table 1 provides a summary of the fabrication method used to make amorphous and anatase forms of the nanotubes. The morphology of the obtained nanotubes was examined by scanning electron microscopy (SEM), with results shown in FIG. 6.

TABLE 1

| TNT/Ti (Amorphous) | TNT/Ti (Anatase) |
|---|---|
| Double anodized Ti metal | Double anodized Ti metal foil |
| foil containing TNT was | containing TNT was dried in air at |
| dried in air at 100° C. | 100° C. for 1 hour; and Annealed at |
| for 1 hour | 500° C. in presence of O₂ for 3 hours |

### Example 3

In this example, TiO₂ nanotubes functionalized with a polyaniline polymer were made. The TiO₂ nanotubes were prepared according to the anodization procedure of Example 2. Then, the TiO₂ nanotubes were dipped into an acetone-based solution comprising 0.4 uM polyaniline and 0.5 M H₂SO₄. The solution was stirred at speed of 200 rpm for 20 hours. In some other embodiments, the nanotubes are immersed in the acetone-based solution for less than 20 hours (e.g., 1 to 5 hours) and then the process is repeated a second time. The sample was then rinsed in DI water. Electrodeposition was carried out in the same solution at a constant potential of-1 V for 10 minutes. The sample was then washed with DI water followed by ethanol and dried under air flow.

### Example 4

In this example, a uniform polyaniline (“PANi”) layer was deposited on the top of a nanotube array wherein the nanotubes were in the anatase form. The anatase-form TiO₂ nanotubes were prepared according to the anodization procedure of Example 2. The PANi layer was deposited using a potentiostatic method at a constant potential of 1.5 V for 400 seconds in an electrolyte solution containing 0.2 M aniline and 0.5 M sulphuric acid (H₂SO₄). The sample was then immersed in 0.5M H₂SO₄ acid for 5-10 minutes, followed by rinsing and washing with ethanol before drying in a vacuum oven at 60° C. for 24 hours. FIG. 3 shows a representation of the camera images of the nanotube array before and after electrodeposition of the PANi layer.

### Example 5

In this example, TiO₂ nanotubes functionalized with aminopropyl triethoxysilane (“APTES”) were made. The TiO₂ nanotubes were prepared according to the anodization procedure of Example 2 and were coated with the APTES in both amorphous form and anatase form. Separately, the amorphous and anatase forms of the TiO₂ nanotubes were incubated overnight in an acetone-based solution comprising 5% (v/v) APTES solution. The samples were then removed from the solutions and briefly sonicated. The samples were then rinsed with acetone and dried at 60° C. for 2 hours.

### Example 6

In this example, a layer of antibodies was immobilized onto functionalized TiO₂ nanotubes comprising either PANi or APTES to provide a coating comprising the PANi (or APTES) and the antibody on the nanotubes. In this example, a drop casting technique was used. The functionalized nanotubes comprising the APTES surface binding agent were prepared according to Examples 5 and the functionalized nanotubes comprising the PANi surface binding agent were prepared according to Example 4. After deposition of the surface binding agent, and prior to any washing step, the functionalized nanotubes were exposed to a coupling solution comprising the antibody, EDC, and NHS by drop casting the coupling solution onto the functionalized nanotubes. For the solution comprising the antibody, 100 μL of a 5 μg/mL antibody solution was added with 1:2 (molar ratio) mixture solution of EDC and NHS in 1 mL ultrapure water. The antibody was a commercial antibody that recognizes s-RBD. The resulting mixture was then left for 1hour at room temperature prior to use. After depositing the antibody solution, the samples were allowed to incubate at ambient temperature for 2 hours to form the coating. The samples were then rinsed with ultrapure water (e.g., five times) to remove any unbound antibody from the surface. Then, 30 uL of a bovine serum albumin (BSA) solution (10 mg/mL) was dropped cast onto the samples and allowed to incubate for 30 minutes. A second drop casting of BSA was then used, to ensure blockage any non-specific adsorption sites on the sample. The synthesized substrate-based platforms from this example are shown in FIGS. 2A-2C.

### Example 7

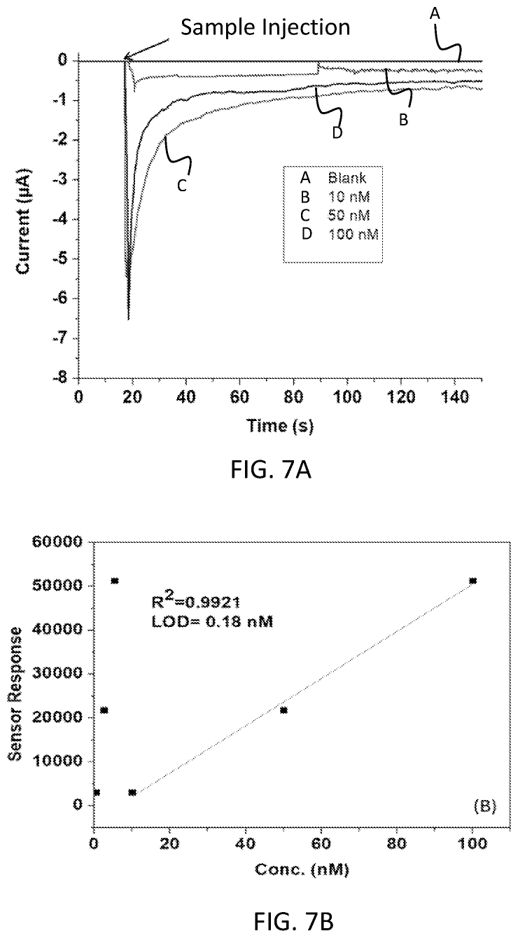

In this example, the ability of an embodiment from Example 6 (nanotube array comprising a coating of PANi and antibody) to detect s-RBD, which is a biomarker protein for COVID-19, was evaluated. Chronoamperometric analyses of different concentrations of the s-RBD protein were performed at a bias voltage of-0.6 V for 150 seconds. A sample volume of 3 μL containing s-RBD protein in ultrapure water was pipetted on the substrate-based platform at 15 seconds after the scanning started in the potentiostat. The sensor response current was observed to increase rapidly as the s-RBD protein conjugated with the antibody to cause an electrochemical change in the sensor. It was observed that the peak current increased (became more negative) from −0.77 at 10 nM of protein to −5.4 and −6.5 pA for 50 and 100 nM of protein, respectively, as shown in FIG. 7A. The average sensor response time was calculated to be ˜1.5 seconds. The sensor response was calculated from the following equation:

$$Sensor Resposne ( S R ) = \frac{{i}_{max , biomarker} - {i}_{max , base line}}{{i}_{max , base line}}$$

The value of i_{max,base line}, which is the current noted when the sensor is not exposed to the s-RBD protein was found to be ˜3.24×10⁻⁵ uA. The calculated sensor responses for 10, 50 and 100 nM were 3.0×10³, 2.19×10⁴, and 5.13×10⁵, respectively. A plot of concentration versus sensor response was found to be linear with a correlation coefficient of R²=0.9921 as shown in FIG. 7B. The detection limit was calculated to be 0.18 nM.

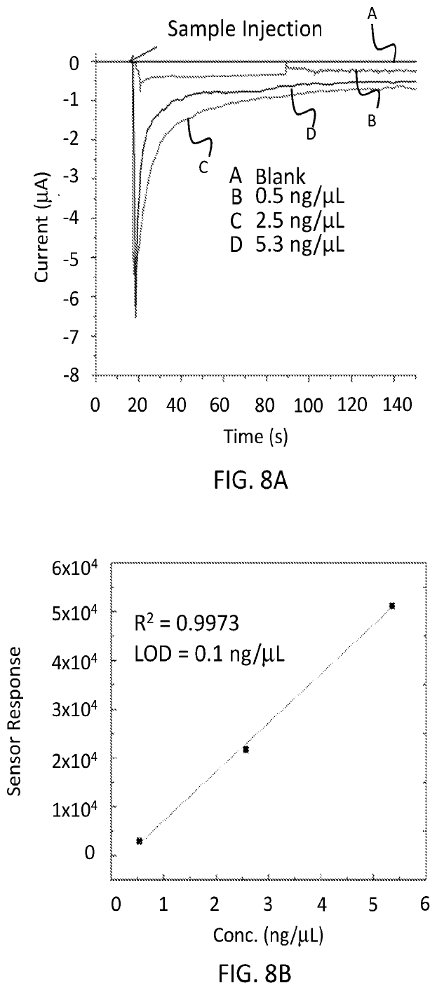

The amperometric response of the sensor (see FIG. 8A) and limit of detection was also calculated in ng/uL unit. A plot of concentration of 0.5, 2.5 and 5.3 ng/uL versus sensor response was found to be linear with a correlation coefficient of R²=0.9973 as shown in FIG. 8B. The detection limit was calculated to be 0.1 ng/uL.

### Example 8

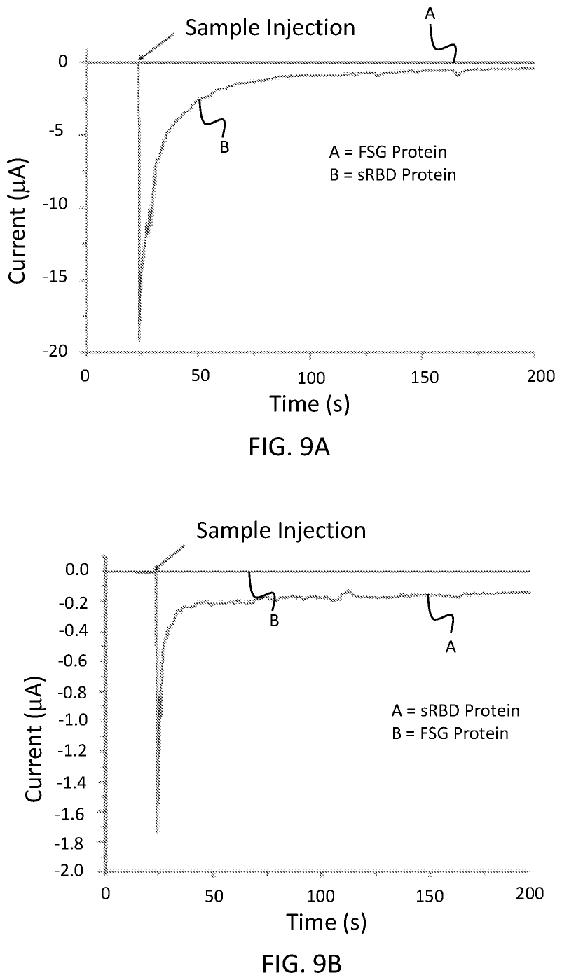

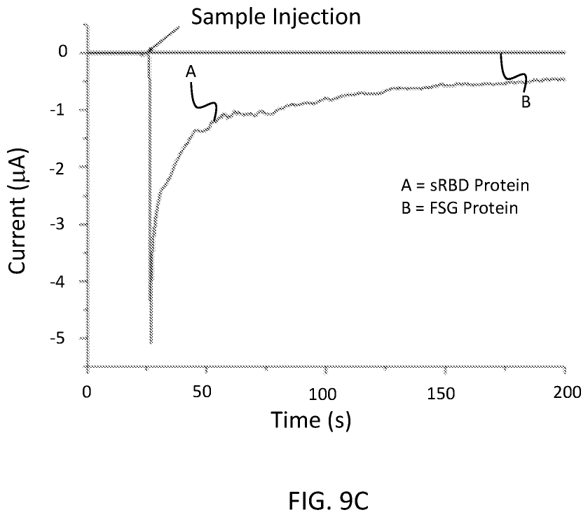

In this example, all three sensors from Example 6 (namely, (i) AB/APTES/TNT (amorphous)/Ti, (ii) AB/APTES/TNT (anatase)/Ti, and (iii) AB/PANI/TNT (anatase)/Ti, wherein AB =antibody, TNT =TiO2 nanotubes, and Ti =titanium substrate) were evaluated for specificity using a specificity test in a similar setup that was used for the electrochemical measurement of amperometric detection of s-RBD protein on AB/PANI/TINT sensor described in Example 7. The attached antibody is specific to capture the target s-RBD protein of covid-19. A different protein, focal glomerulosclerosis (FSG), was also tested along with s-RBD on the devices to check specificity towards the only s-RBD of Covid-19. It was found that all three sensors responded instantly after pipetting 3 uL of 50 nM or 2.5 ng/uL s-RBD on the sensor at the 20th second, as shown in FIGS. 9A-9C. The sensors were not responsive to FSG (FIGS. 9A-9C) after being introduced on the sensor at 20th second during the amperometric scan period of 200 seconds.

Example 9

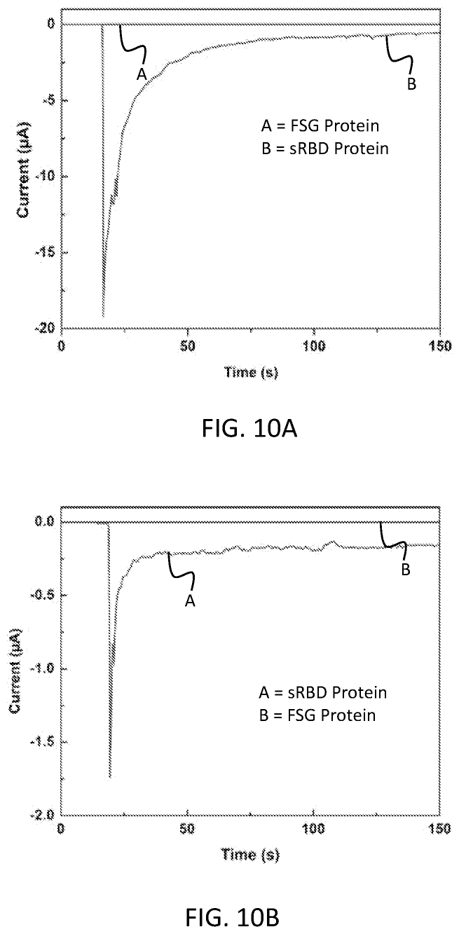

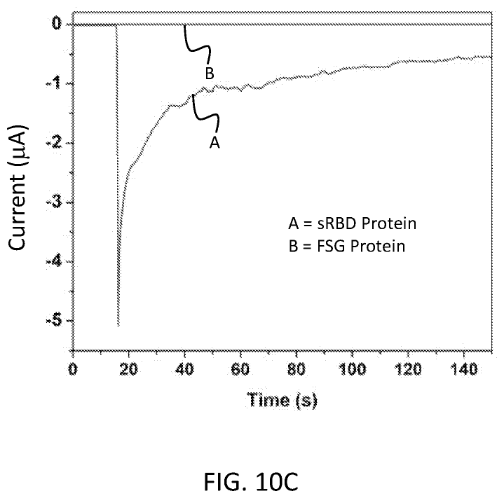

In this example, a specificity test of the sensors was performed to assess whether the sensors may encounter other proteins within the same sample domain of actual samples. Focal glomerulosclerosis (FSG) protein along with sRBD was prepared and analyzed to investigate the specificity of the following three sensors chips: (a) ab-APTES/TNT (amo)/Ti, (b) ab-APTES/TNT (ana)/Ti, and (c) ab-PANI/TNT (ana)/Ti. While FSG is not a viral protein, it was chosen to compare the ability of the proposed sensor to distinguish and detect sRBD amongst other proteins and to evaluate the specificity of the immobilized anti-SARS-COV2-sRBD on sensor chips towards capturing sRBD of SARS-COV2 only. It was determined that all three sensors responded instantly with the detectable current after introducing 5 pL of sRBD protein on the sensor at the 15th second, as shown in FIGS.10A-10C. The sensors were not responsive to FSG (FIGS. 10A-10C) after being introduced on the sensor at 15th second during the amperometric scan period of 150 seconds.

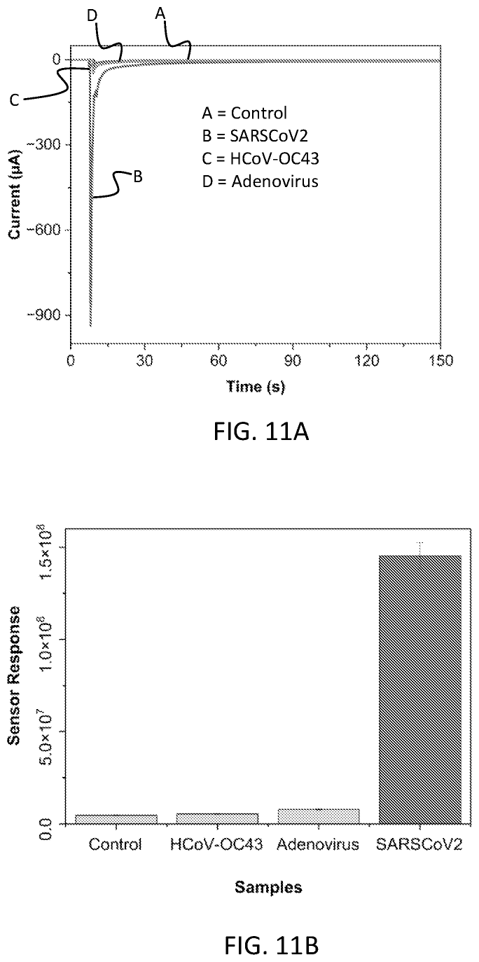

Specificity was further confirmed by analyzing SARSCOV2, HCoV-OC43, and adenovirus on the ab-PANI/TNT (ana)/Ti sensor. In this case, sensors were exposed to 10 ul of the control sample (Saliva), SARSCOV2, HCoV-OC43, and adenovirus for 5 minutes. The exposed sensors were then washed with PBS buffer to remove any unconjugated virus or protein from the immobilized antibody layer of the sensors prior to chronoamperometry (CA), cyclic voltammetry (CV) and electrochemical impedance spectroscopy (EIS) scans in presence of PBS buffer. CA signal in FIG. 11A indicated that the surface-immobilized antibody (anti-SARSCOV2) of the sensor was able to selectively conjugate/hybridize the SARSCOV2 virus. A strong CA signal was observed from SARSCOV2 samples in comparison with HCoV-OC43 or adenovirus.

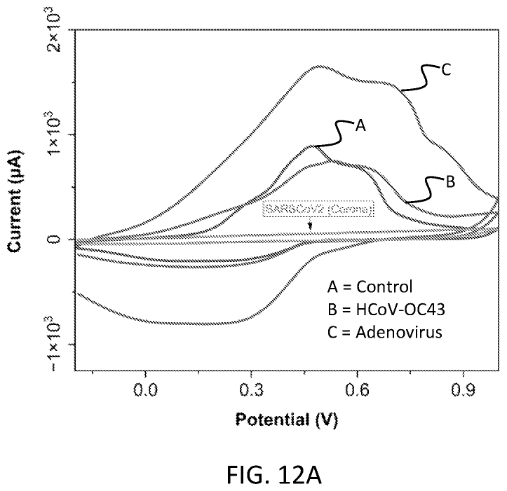

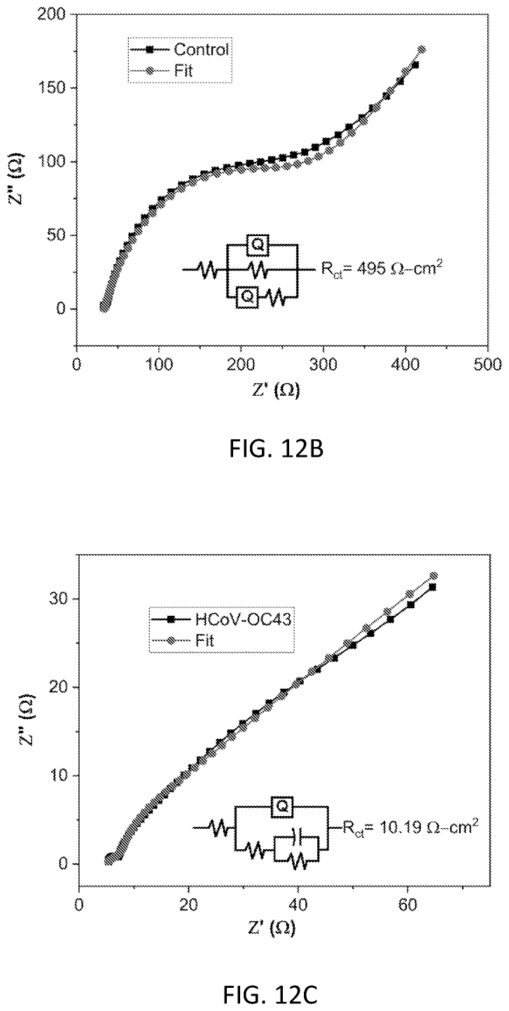

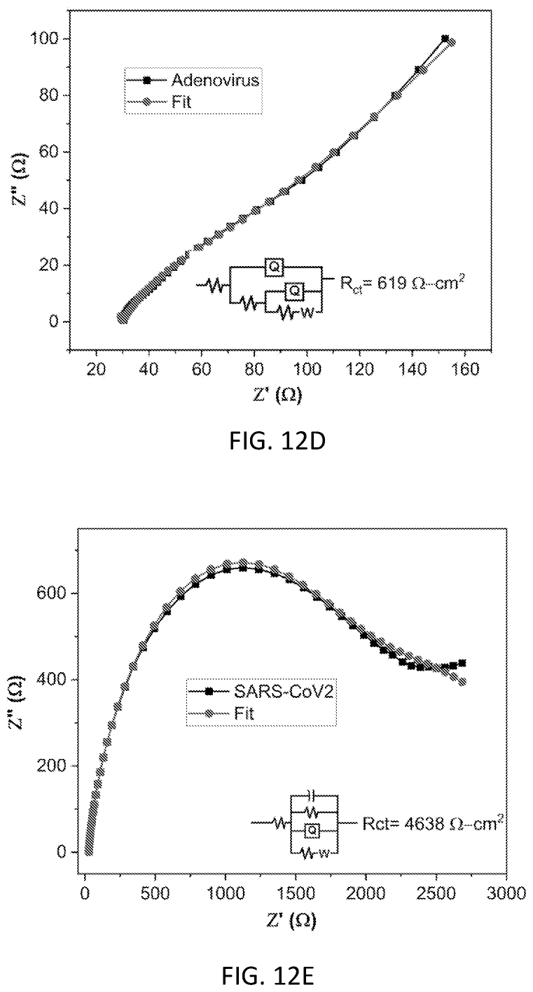

Sensor response was calculated from the CA data in FIG. 11A, which clearly demonstrated that the proposed sensor is highly selective toward SARSCOV2 virus (see FIG. 11B). This observation was further justified by corresponding CV and EIS analysis of all the above-mentioned samples. CV scanning-shown in FIG. 12A-revealed suppressed sensor conductivity for the SARSCOV2 sample in comparison with saliva (control), HCoV-OC43, and adenovirus because of the deceleration of electron transfer due to hybridization of target viruses with antibody. These results indicated the presence of SARSCOV2 on the sensor surface through antibody-antigen conjugation/hybridization even after washing with PBS buffer. Viruses other than SARSCOV2 rarely existed on the sensor surface, likely because of the nature of the specificity of the antibody that only recognizes SARSCOV2. A similar phenomenon was observed in the EIS data. At the same setup of CV, the readings of EIS indicated an increase in impedance for the SARSCOV2 sample. A charge transfer resistance order after the conjugation/hybridization on the sensor was found as SARSCOV2>Saliva/HCoV-OC43/adenovirus. The data was best fitted with the modified equivalent circuit shown in FIG. 12A, with results shown in FIGS. 12B-12E. The equivalent circuit comprises solution resistance (Rs), charge transfer resistance (Rct), Capacitor @, Warburg element (W) and a constant phase element (CPE). Rct was determined to be 495, 10.19, 619, and 4638 0 for saliva (Control), HCoV2-OC43, adenovirus, and SARSCOV2, respectively. The data indicated that the highest Rct of 4638 Q was obtained from SARSCOV2 samples. This might be happened due to the increased antigen (SARSCOV2)-antibody binding that decreased the mass transfer flux of the electrolyte toward the electrode. These data establish that sensor embodiments according to the present disclosure are sensitive and selective for SARSCOV2.

In view of the many possible embodiments to which the principles of the present disclosure may be applied, it should be recognized that the illustrated embodiments are only preferred examples and should not be taken as limiting the scope of the present disclosure. Rather, the scope is defined by the following claims. We therefore claim as our invention all that comes within the scope and spirit of these claims. Listing of Claims

1. An electrode, comprising a plurality of functionalized nanotubes on a support, wherein the functionalized nanotubes comprise metal oxide-based nanotubes that are associated with a coating comprising a surface binding agent and biomolecule.

2. The electrode of claim 1, wherein the metal oxide-based nanotubes of the plurality of functionalized nanotubes have an average length greater than 3 um and wherein metal oxide-based nanotubes comprise a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof.

3. (canceled).

4. The electrode of claim 1, wherein the metal oxide-based nanotubes are TiO2 nanotubes that exist in anatase form, amorphous form, or a combination thereof.

5. The electrode of claim 1, wherein the surface binding agent is an amino-containing compound, a carboxylic acid-containing compound, a maleimide-containing compound, a haloacetyl-containing compound, a pyridyldithiol-containing compound, or a combination thereof.

6. The electrode of claim 5, wherein the amine-containing compound is a polyaniline polymer having average Mw ranging from 1,000 to 100,000 or a salt thereof; an amino-silane compound selected from aminopropyl triethoxysilane, 3-aminopropyldimethylethoxysilane, 3-aminopropyltrimethoxysilane, propyldimethylmethoxysilane, or N-(6-aminohexyl) aminomethyltriethoxysilane; or a combination thereof.

8. aims 7 and 8 (Canceled).

9. The electrode of claim 1, wherein the biomolecule is capable of specifically binding an antigen.

10. The electrode of claim 9, wherein biomolecule is an antibody that specifically binds a COVID-19 antigen.

11. (canceled).

12. The electrode of claim 10, wherein the antibody is covalently bound to the surface binding agent such that antigen-binding regions of the antibody extend vertically upwards and wherein the surface binding agent is coated on top of the functionalized nanotubes.

13. The electrode of claim 1, wherein the electrode further comprises a blocking agent that covers regions of the functionalized nanotubes and/or surface binding agent that do not further comprise a biomolecule and/or wherein the support is a metal support.

14. (canceled).

15. The electrode of claim 1, further comprising a substrate physically attached to the support of the electrode, wherein the substrate comprises a fiber-based material, a plastic material, a glass material, or a metal material.

16. (canceled).

17. A substrate-based platform comprising:
the electrode of claim 1; and
a substrate physically attached to the support of the electrode.

18. The substrate-based platform of claim 17, wherein the functionalized nanotubes comprise TiO2-based nanotubes functionalized with a coating comprising (i) a layer of a surface binding agent selected from a polyaniline polymer, an amino-silane compound, or a combination thereof; and (ii) an antibody.

19. The substrate-based platform of claim 18, wherein the antibody is covalently bound to the layer of the surface binding agent and the coating covers the plurality of functionalized nanotubes.

20. An electrochemical biomolecule-functionalized sensor device, comprising:
a working electrode component, comprising the electrode of claim 1;
a reference electrode;
a counter electrode; and
a potentiostat.

21. The electrochemical biomolecule-functionalized sensor device of claim 20, further comprising a power source; a sample introduction inlet or region; a housing; or any combination thereof.

22. (canceled).

23. A method, comprising:
applying a voltage to the electrochemical biomolecule-functionalized sensor device according to claim 20;
exposing the electrochemical biomolecule-functionalized sensor device to a biological sample by contacting the working electrode or the substrate-based platform of the sensor device with the biological sample; and
sensing a change in current produced by the electrochemical biomolecule-functionalized sensor device after being exposed to the biological sample.

24. (canceled).

25. The method of claim 23, further comprising measuring the change in current produced by the electrochemical biomolecule-functionalized sensor device and wherein the change in current produced by the electrochemical biomolecule-functionalized sensor device signifies a binding event between a biological indicator present in the biological sample and biomolecule of the working electrode or the substrate-based platform.

26. (canceled).

27. The method of claim 23, wherein the biological indicator is an antigen, a bacterium, a virus, or a cellular component thereof.

28. The method of claim 23, wherein the biological indicator is SARS-COV-2 spike protein and the biomolecule is an antibody.

29. The method of claim 23, wherein the method further comprises diagnosing a subject from which the biological sample is obtained, wherein the subject has, or is at risk of developing, a physiological condition or disease and wherein the disease is COVID-19.

30. (canceled).

31. The method of claims 23, wherein the voltage is applied to the sensor device using a power source that is integrated in the electrochemical biomolecule-functionalized sensor device or that is an external power source.

32. (canceled).

33. The method of claim 23, wherein the biological sample is a saliva sample, a nasal mucous sample, a breath sample, or a combination thereof.

34. A method of making the electrode of claim 1, comprising:
performing a first anodization of the support to obtain the metal oxide-based nanotubes formed thereon;
performing a second anodization of the metal oxide-based nanotubes to increase the length of the metal oxide-based nanotubes;
depositing the surface binding agent on the metal oxide-based nanotubes to form a layer of the surface binding agent on surfaces of metal oxide-based nanotubes; and
depositing a solution comprising the biomolecule onto the layer of the surface binding agent.

35. The method of claim 34, wherein the solution comprising the biomolecule further comprises one or more coupling reagents and/or wherein the method further comprises depositing a blocking agent after depositing the solution comprising the biomolecule.

36. The method of claim 34, wherein depositing the surface binding agent comprises using an electrochemical deposition technique or a drop deposition technique and wherein depositing the solution comprising the biomolecule comprises using a drop deposition technique.

38. ims 37 and 38 (Canceled).
